(19) United States
(12) Patent Application Publication
(10) Pub. No.: US 2010/0258137 A1
(43) Pub. Date: Oct. 14, 2010

(54) DEVICE AND METHOD FOR PREVENTING OR REDUCING SNORING AND/OR SLEEP APNEA

(76) Inventor: Ivan James Scott, Werribee (AU)

Correspondence Address: BIO INTELLECTUAL PROPERTY SERVICES (BIO IPS) LLC, 8509 KERNON CT., LORTON, VA, 22079, US

(21) Appl. No.: 12/755,653

(22) Filed: Apr. 7, 2010

(30) Foreign Application Priority Data
Apr. 8, 2009 (AU) 2009201383

(51) Int. Cl. A61F 5/56

(52) U.S. Cl. 128/848

(57) ABSTRACT

A device is used to prevent or lessen the likelihood of a person, prone to snoring and/or sleep apnoea, from experiencing those conditions. The device includes a back-sleep-preventer that keeps the person substantially in a front-sleep position lying on a bed proximate an edge of the bed. The hack-sleep-preventer restricts the person to the front-sleep position to the extent of preventing the person from thereafter rolling away from the edge of the bed to adopt a back-sleep position. This prevents or at least lessens the likelihood of person experiencing snoring and/or sleep apnoea. Anchor-means releasably secures the back-sleep-preventer to the bed-means. The anchor-means is connected to the person or the person's upper garment by a connector-means. When the person gets off and moves away from the bed-means, with the connector-means still connecting the person or the person's garment to the anchor-means, the anchor-means is able to release the back-sleep-preventer from the bed-means.

### CROSS-REFERENCE TO RELATED APPLICATIONS

This application claims the benefit of Australian Application No. 2009201383, filed Apr. 8, 2009. The entire disclosure of the prior application is hereby incorporated by reference.

### FEDERALLY SPONSORED RESEARCH: not applicable

### SEQUENCE LISTINGOR PROGRAM: not applicable.

### FIELD OF INVENTION

The present invention relates to a device & method for preventing or reducing snoring and/or sleep apnea.

### BACKGROUND OF THE INVENTION

There are known devices that address the problem of snoring and/or the condition known as sleep apnea, which is characterised by a person having pauses in breathing during sleep.

Some approaches to addressing such sleep disorders require a person to insert a device into the mouth. This can be uncomfortable and may interfere with the person's sleep to a large degree.

Other approaches require a device to be fitted in or to the person's nose which can also be uncomfortable for the person and can tend to interfere with sleep to a large degree.

These and other devices in the prior art can tend to be complex to use and/or manufacture, or can also tend to interfere with sleep to a large degree.

An object of the present invention is to overcome or at least ameliorate one or more of the problems in the prior art, or to provide an improved alternative.

### SUMMARY OF THE INVENTION

According to the present invention, there is provided a device for preventing or lessening the likelihood of a person, prone to snoring and/or sleep apnoea, from experiencing the same, comprising:

a back-sleep-preventer adapted to ensure that, in use when a person lies on a bed-means initially in a front-sleep position and proximate an edge of the bed-means, the back-sleep-preventer is adapted to be arranged in a manner to substantially restrict the person to the front-sleep position to the extent of preventing the person from thereafter rolling away from the edge of the bed-means to adopt a back-sleep position such that, if the person is prone to snoring and/or sleep apnoea, then being prevented from adopting the back-sleep position by the back-sleep-preventer is able to prevent or at least lessen the likelihood of person experiencing snoring and/or sleep apnoea, wherein the back-sleep-preventer includes:

anchor-means able to releasably secure at least part of the back-sleep-preventer to the bed-means; and

person-to-anchor-connector-means adapted to connect the person or the person's upper garment to the anchor-means in order to substantially restrict the person to the front-sleep position in said aforesaid manner;

and wherein, in use when the person gets off and moves away from the bed-means, with the person-to-anchor-connector-means still connecting the person or the person's garment to the anchor-means, the anchor-means is able to release the at least part of the back-sleep-preventer from the bed-means.

Preferably, in use when the bed-means includes a mattress or mat, the anchor-means is adapted to be inserted underneath the mattress or mat in order to substantially resist the pull of the person's rolling motion on the mattress or mat to restrict the person to the front-sleep position.

Preferably, the anchor-means includes weight-enhanceable-anchoring-means such that, in use when the person is lying on the mattress or mat with the person's body weight acting on the weight-enhanceable-anchoring-means, the weight-enhanceable-anchoring-means is enhanced in its ability to act as an anchor to the extent that the person's body weight and the weight of the bed-means makes it more difficult for the anchor-means to be disconnected from the bed-means.

Preferably, the weight-enhanceable-anchoring-means includes a flat portion that is adapted, in use, to be inserted underneath mattress or mat'such that, in use when the person lies on the mattress or mat in a position that is above the flat portion, the person's body weight and/or the mattress weight are able to press on the flat portion thereunderneath to enhance the ability of the flat member to resist being pulled out from beneath the mattress or mat.

Preferably, in use when the person gets off and moves away from the bed-means, with the person-to-anchor-connector-means still connecting the person or the person's garment to the flat portion, the flat portion is able to be pulled out from underneath the mattress or mat in order to release the at least part of the back-sleep-preventer from the bed-means.

The person-to-anchor-connector-means may connect to a lateral side of the person or of the person's garment.

Alternatively, the person-to-anchor-connector-means may connect to a lateral side of the person's upper garment.

Preferably, the person-to-anchor-connector-means is able to be arranged to have sufficient length to restrict the person to sleeping in the initial front-sleep position, and to be arranged so as to have insufficient length that would otherwise permit the person, from the initial front-sleep position, to subsequently adopt a back-sleep position.

Preferably, the person-to-anchor-connector-means functions as a leash having a leash-length that defines the outermost extent to which the person is permitted by the leash to move or roll away from the edge of the bed-means and the anchor-means.

Preferably, the person-to-anchor-connector-means includes leash-length-adjustment-means that allows length-adjustment of the leash-length of the person-to-anchor-connector-means.

Preferably, the person-to-anchor-means-connector-means includes one or more straps that arc adjustable in length.

According to another aspect of the present invention, there is provided a method of preventing or lessening the likelihood of a person prone to snoring and/or sleep apnoea from experiencing the same, the method including:

restricting a person to a front-sleep position, when the person lies on a bed-means initially in a front-sleep position and proximate an edge of the bed-means, by arranging a back-sleep-preventer in a manner to substantially restrict the person to the front-sleep position to the extent of preventing the person from thereafter rolling away from the edge of the bed-means to adopt a back-sleep position such that the person being prevented from adopting the back-sleep position by the back-sleep-preventer is able to prevent or at least lessen the likelihood of person experiencing snoring and/or sleep apnoea,

wherein the method includes using person-to-anchor-connector-means of the back-sleep-preventer to connect the person or the person's upper garment to anchor-means of the back-sleep-preventer;

and also includes using the anchor-means to releasably secure at least part of the back-sleep-preventer to the bed-means in order to substantially restrict the person to the front-sleep position in said aforesaid manner, such that, when the person gets off and moves away from the bed-means, with the person-to-anchor-connector-means still connecting the person or the person's garment to the anchor-means, the method includes the anchor-means releasing the at least part of the back-sleep-preventer from the bed-means.

In a preferred embodiment, the bed-means includes a mattress or mat, and the method preferably includes inserting the anchor-means underneath the mattress or mat to releasably secure at least part of the back-sleep-preventer to the bed-means.

Preferably, the anchor-means includes weight-enhanceable-anchoring-means and wherein the method includes the person lying on the bed-means with the person's body weight acting on the weight-enhanceable-anchoring-means to allow the weight-enhanceable-anchoring-means to enhance its ability to act as an anchor to the extent that the person's body weight and the weight of the mattress makes it more difficult for the anchor-means to be disconnected from the bed-means.

Preferably, the weight-enhanceable-anchoring-means includes a flat portion, and wherein the method preferably includes inserting the flat portion underneath the mattress of the bed-means such that the person lying on the mattress in a position above the flat portion causes the person's body weight and/or the mattress weight to press on the flat portion thereunderneath to enhance the ability of the flat member to resist being pulled out from beneath the mattress.

Preferably, when the person gets off and moves away from the bed-means, with the person-to-anchor-connector-means still connecting the person or the person's garment to the flat portion, the method preferably includes pulling the flat portion out from underneath the mattress or mat in order to release the at least part of the back-sleep-preventer from the bed-means.

Preferably, the method includes connecting the person-to-anchor-connector-means to a lateral side of the person, or to a lateral side of the person's upper garment.

Preferably, the method includes arranging the person-to-anchor-connector-means to have sufficient length to restrict the person to sleeping in the initial front-sleep position, and arranging the person-to-anchor-connector-means to so as to have insufficient length that would otherwise permit the person, from the initial front-sleep position, to subsequently adopt a back-sleep position.

Preferably, the method includes using the person-to-anchor-connector-means as a leash having a leash-length that defines the outermost extent to which the person is permitted by the leash to move or roll away from the anchor-means.

### DRAWINGS

In order that the present invention might be more fully understood, embodiments of the invention will be described, by way of example only, with reference to the accompanying drawings, in which:

FIG. 1 is a schematic end view of a person lying down on a mattress in what will be referred to herein as a hack-sleep position;

FIGS. 2A and 2B are simplified representations of schematic end views of a person, using a preferred embodiment of the present invention which prevents the person, initially in a front-sleep position of FIG. 2A, from thereafter adopting a back-sleep position, where:

FIG. 2A is a simplified schematic end view of the person lying down on the mattress initially in a front-sleep position, and

FIG. 2B shows the person attempting to roll out of the front-sleep position, but being prevented, by the embodiment, from rolling into a back-sleep position;

FIG. 3A is a side, perspective view equivalent to the schematic FIG. 2A showing the person using the embodiment while lying initially in the front-sleep position;

FIG. 3AA is a plan view of FIG. 3A;

FIGS. 4A and 4B show an action sequence of the user (the person) arranging the embodiment so as to enable it to prevent himself from subsequently rolling into a back-sleep position;

FIG. 5 shows the embodiment of the invention attached to a lateral side of a shirt that can be worn by the person;

FIG. 6 shows an illustration of the person adjusting the length of the strap in the embodiment;

FIG. 7 shows an illustration of an adjustor buckle of the strap in the embodiment; and

FIG. 8 shows a modified embodiment used by a person lying on a thin mat.

### DESSCRIPTION OF THE EMBODIMENTS

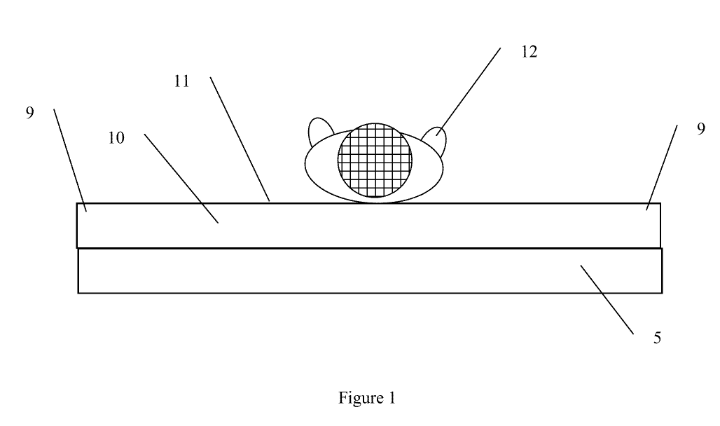

In the accompanying drawings, FIG. 1 shows a diagram of a person sleeping on a bed-means which, in this embodiment, comprises a bed with a mattress on top of a base. In other embodiments, the bed-means can be in the form of a range of other objects that a person lies on to sleep, such as a portable mattress, inflatable mattress, a sleeping mat, a futon etc.

In FIG. 1, the person is in a back-sleep position with his back lying on the upper surface 11 of a mattress 10 of a bed.

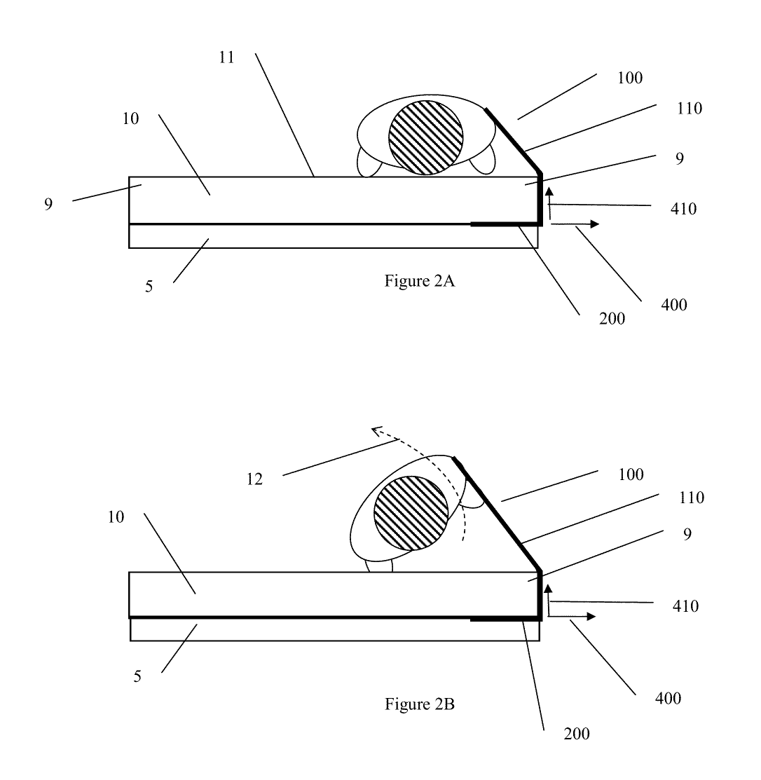

(In the diagrams of FIGS. 1 and 2A and 2B, the person's feet 12 provide an indication of the direction in which the person's chest is facing).

In contrast to FIG. 1, the diagram of FIG. 2A shows another person sleeping initially in a front-sleep position with his chest and stomach facing flat on the surface 11 of the mattress 10 of the bed. In this example, the person is prone to suffering from a condition of sleep apnea and is prone to snoring.

Without being restricted by theory, the present invention assumes that a person's snoring and/or sleep apnea can be exacerbated by sleeping in a back-sleep position, for example in FIG. 1.

Also, without being restricted by theory, another proposition of the present invention is that there is a greater likelihood that the person's snoring and/or sleep apnea can be prevented or at least substantially lessened if the person were to sleep in the front-sleep position, as indicated in FIG. 2A. Thus, according to the embodiment, either or both of those conditions can be prevented, or at least the likelihood of the person experiencing snoring and/or sleep apnoea can be lessed, by the person being prevented from adopting the back-sleep position by the device of the embodiment.

Thus, an embodiment of the invention is designed to prevent the person, who sleeps initially in a front-sleep position, from thereafter rolling or otherwise moving away from the edge of the bed into a back-sleep position. In other words, according to the above propositions, the back-sleep position of FIG. 1 is to be strictly avoided with the aid of the embodiment of the invention.

### GENERAL CONCEPTS

In order to explain general concepts of the embodiment of the invention, reference is made to the schematic diagrams of FIGS. 2A and 2B.

FIGS. 2A and 2B show an embodiment of a back-sleep-preventer in the form of a strap device 100. In these FIGS. 2A and 2B, the strap device 100 is shown schematically as a straight line merely for the sake of demonstrating the overall concept of how the embodiment functions.

FIG. 2A shows a person sleeping initially in a front-sleep position proximate the edge 9 of a bed.

FIGS. 2A and 2B shows how the person makes use of a strap device 100 to prevent himself from ever subsequently rolling over, away from the edge 9 of the bed, into a back-sleep position of FIG. 1. In other words, the person commences his bed time in the front-sleep position of FIG. 2A, and the strap device 100 prevents the person from ever rolling over into the back-sleep position of FIG. 1.

FIG. 2B shows the person, who had initially been in the front-sleep position proximate the edge 9 of the bed, attempting to roll away from the edge 9, out of that front-sleep position (the rolling motion is indicated by arrow 12), however, the strap device 100 restrains the person from substantially leaving the front-sleep position. In FIG. 2B, once the strap device 100 halts the rolling motion 12 when the person reaches about 45 degrees from the horizontal, the person rolls or falls back into the front-sleep position. Specifically, the person is prevented by the strap device 100 from entering the back-sleep position.

In other words, the strap device 100 is able to ensure that, when a person lies on a bed initially in a front-sleep position proximate an edge 9 of the bed, the strap device 100 is able to be arranged to substantially restrict the person to the front-sleep position to the extent of preventing the person from thereafter rolling away from the edge of the bed to adopt a back-sleep position.

A feature of the strap device 100 is that, if the person is prone to snoring and/or sleep apnoea, then, according to the stated propositions, using the strap device 100 to prevent the person from adopting the back-sleep position is able to prevent or at least lessen the likelihood of the person snoring and/or experiencing sleep apnoea.

### Resistance To Rolling Away From The Edge

In summary, in order for the strap device 100 to resist the person's attempt to roll away from the edge of the bed-means, into the back-sleep position, part of the strap device 100 is connected, in some manner, to the person. The concept of being connected to the person includes connection to the person's body or to the person's clothes. In either case, the strap device 100 is able to exert a pulling force on the person's body that resists any attempts to roll into the back-sleep position.

### (i) Attachment To The Person

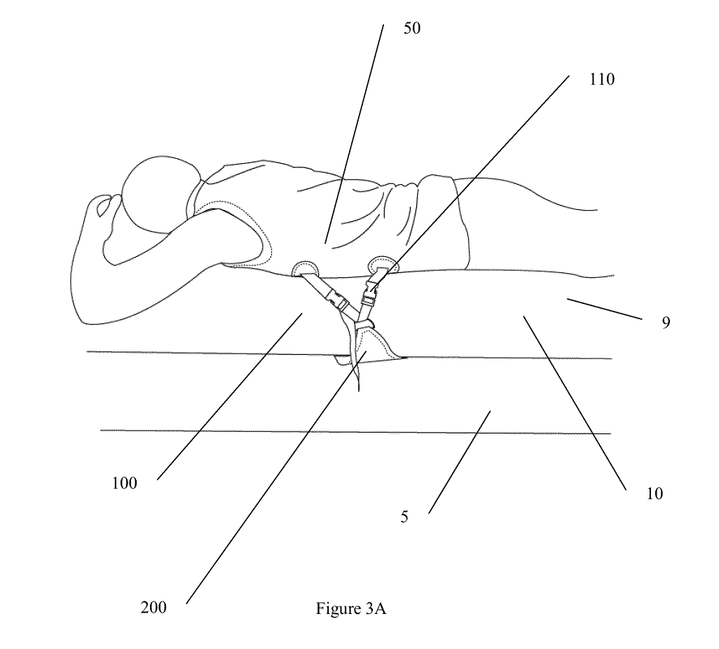

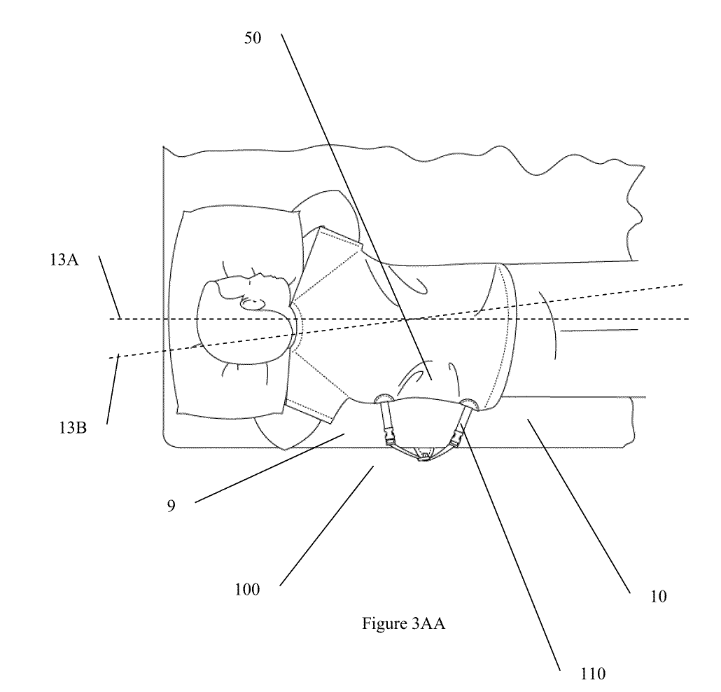

In the embodiment of FIGS. 3A to 5, part of the strap device 100 is preferably secured to the lateral side 50 of the person's upper garment 54.

The lateral side is the preferred location because such an arrangement prevents the person from acquiring the back-sleep position by axially rotating, which could happen if the strap device 100 were to be attached, perhaps, to the middle of the person's back.

The upper garment may be a shirt, T-shirt, blouse, pyjama top, or other known upper garments.

In other embodiments, part of the strap device 100 may conceivably be attached to the lateral side of the person's waist or hip region.

In other embodiments, if the person sleeps without an upper garment, modifications of the strap device can physically attached to the person by wrapping around the person's torso, however, in such modifications, the strap device preferably still makes a point of connection with the lateral side of the person's body.

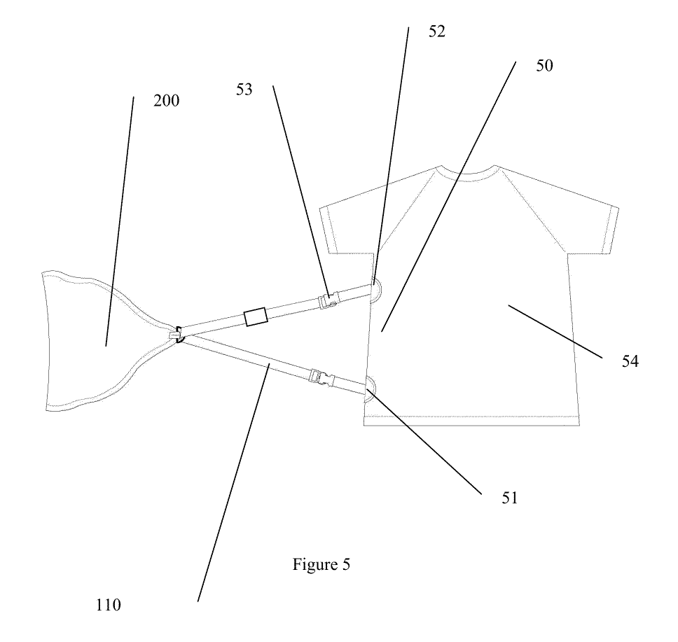

Referring to FIG. 5 as an example, the lateral side of an upper garment is understood to be the portion of the garment, when worn, that is proximate the side of a person's torso and waist, in other words, not proximate the person's chest or back.

With reference to the general concepts in FIGS. 2A and 2B, the strap device 100 is able to prevent the person from adopting a back-sleep position because at least part of the strap device 100, during use, is able to be secured to a source of resistance. For example, in this embodiment, the source of resistance is the bed.

Thus, when the person, during sleep or even when awake, attempts to roll towards the back-sleep position, the securement of the strap device 100 to the bed enables the strap device 100 to resist the person's attempt at rolling, at least to the extent that it prevents the person from rolling into the back-sleep position.

A certain amount of rolling, however, is permitted so that the person is not unduly restricted so as to affect his ability to sleep comfortably. For instance, FIG. 2B shows that the strap device 100 has been arranged to have a length that allow the person to roll about 45 degrees which may be sufficient to accommodate slight amounts of body movement during sleep.

### (ii) Attachment To The Bed

In FIGS. 2A and 2B, the bed has a base 5 which has a mattress 10 thereon. The weight of the mattress 10 presses down on the base 5. In use, the mattress is lifted up, and part of the strap device 100 is inserted underneath the mattress. In other words, part of the strap device 100 is laid on top of the base 5, and then the mattress is lowered on top of that part of the strap device 100, so as to sandwich the part of the strap device 100 between the base and mattress.

The part of the strap device 100, that is adapted, in use, to be inserted underneath the mattress 10, includes anchor-means.

The anchor-means includes weight-enhanceable-anchoring-means. In the embodiments of FIGS. 2A and 2B, and also of FIGS. 3A to 5, the weight-enhanceable-anchoring-means is in the form of a flat portion 200. The flat portion 200, in use, is able to be inserted in between the mattress 10 and its base 5 so as to be sandwiched therebetween. In other words, the weight of the mattress makes it difficult to pull the flat portion 200 from out between the mattress and the base. The flat portion 200 generates a higher degree friction when an attempt is made to pull it out from between the mattress and base.

Moreover, when the person lies on top of the mattress 10 with his body weight acting directly on the flat portion 200 that is underneath the mattress, it is even more difficult to pull out the flat portion 200 because of the weight of the person, combines with the weight of the mattress, to make it even more difficult to pull out the flat portion 200 out from between the mattress and base.

The weight of the mattress 10 alone presses on the flat portion 200 to provide resistance, however, the added weight of the person adds pressure onto the flat portion 200 to add further resistance to its removal. Thus, the flat portion 200 is enhanced in its ability to act as an anchor, to the extent that the person's body weight and the weight of the mattress make it more difficult for the anchor-means to be disconnected from the bed.

The flat portion 200 is adapted, in use, to be inserted underneath the mattress of the bed such that, when the person lies on the mattress in a position that is above the flat portion, the person's body weight and/or the mattress weight are able to press on the flat portion thereunderneath to enhance the ability of the flat member to resist being pulled out from beneath the mattress.

In the embodiment of FIG. 5, the sides of the triangle of the flat portion 200 are each around 600 mm, which provides the triangular flat portion with sufficient surface area to act as an anchor when sandwiched underneath the mattress and between its base. In other trials, the flat portion was even found to be effective with sides of around 450 mm. In other modifications, the flat portion can be in the shape of a circle, square, rectangle or other regular or irregular shape. In further modifications, the size dimensions of the flat portion can be either larger or smaller, although the flat portion should not be made so small to a degree that it can no longer offer sufficient anchorage against the pulling force of a person's attempt at rolling on the bed.

In other modifications, the flat portion 200 may be made from a friction-enhancing material such as a coarse fabric, for example, towelling material, hessian or burlap. In other embodiments, the flat portion may even be made of non-textile materials, such as flat sheet rubber or sheet silicon rubber.

In FIGS. 2A and 2B, it can be seen that, when the person lies on the bed, another factor that makes the flat portion difficult to pull out from under the mattress is the fact that the vector direction of the pulling force of the upper part of the strap 110 is in a different direction to the plane of the flat portion 200 underneath the mattress. In other words, in order to pull the flat portion 200 out from between the mattress and the base, it would require a vector force that pulls away from the bed laterally, however, in contrast, the force components of the person pulling, as he rolls away from the edge of the bed, are not aligned in this ideal direction. In FIGS. 2A and 2B, the ideal, lateral removal force 400 that could most easily remove the flat portion 200 out from beneath the mattress would be a force 400 that acts parallel to the plane of the base of the mattress. In contrast, in FIGS. 2A and 2B, the actual force 410, in the embodiment, that arises from the pull of the strap device 100 acts transverse to the ideal removal force 400. This at least partly explains the difficulty in pulling the flat portion from between the bed simply by relying on the rolling motion of the person attempting to roll away from the edge 9 of the bed.

In the embodiment, the flat portion 200 is able to releasably secure at least part of the strap device 100 to the bed such that, in use, when the person gets off and moves away from the bed, the flat portion 200 is able to release that part of the at strap device 100 that was secured to the bed. For example, when the person gets off the bed and walks away with the strap device 100 still attached to his body, the person's motion away from the bed causes the flat portion 200 to be pulled out from underneath the mattress. Thus, for the embodiment, it means that the person can get out of bed and walk away from the bed, specifically without the flat portion 200 holding or locking the person to the bed.

This releasable securement is an advantage of the embodiment because, in the alternative, if the flat portion were to somehow un-releasably secure the person to the bed, a dangerous situation could arise because the person might be unable to escape away from the bed during a fire or other emergency, particularly in the panic of the emergency situation.

### Anchor-Connection

In the embodiment, it is necessary to connect the person to the flat portion 200. This is so that, when the person, initially in a front-sleep position near an edge of the bed, attempts to roll away from the edge into the back-sleep position, the flat portion 200, sandwiched underneath the mattress and between the base, will resist the pull of the person's rolling motion 12. This prevents the person from rolling away from the edge all the way into the back-sleep position.

Hence, to effectively connect the person to the flat portion 200, the strap device 100 includes person-to-anchor-connector-means which, in the embodiment, is in the form of one or more straps 110 that connects the person or the person's upper garment to the flat portion 200.

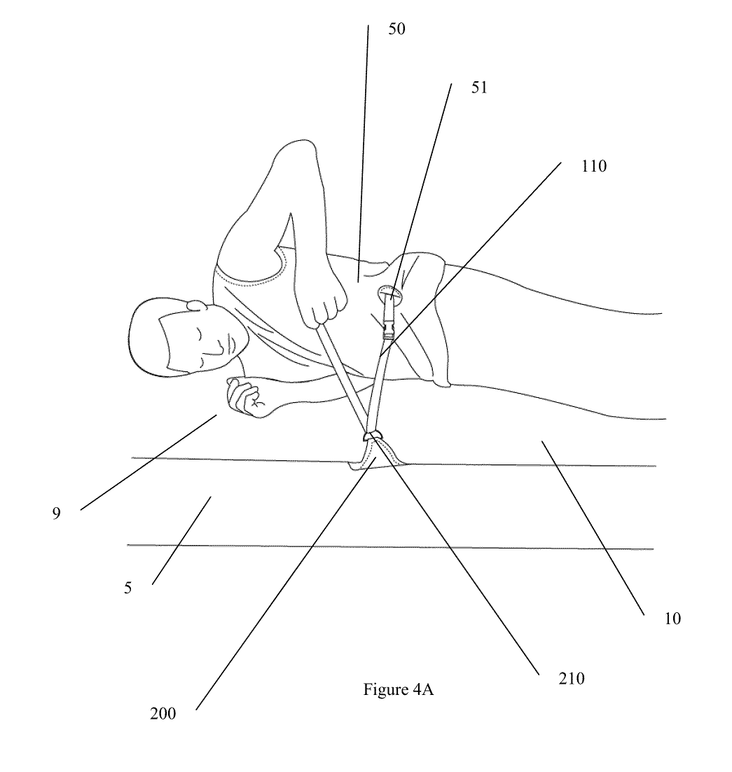

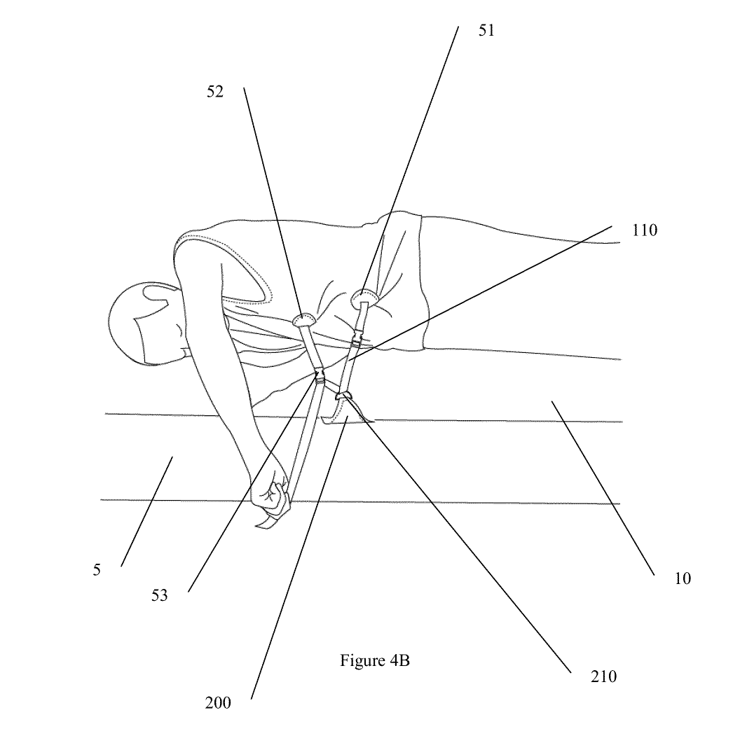

In the embodiment of FIGS. 3A to 5, the person-to-anchor-connector-means consists of one strap 110. The procedure for arranging the strap 110 can he seen in the sequence of FIGS. 4A and 4B.

In FIG. 4A, the strap 110 is connected to a portion 51 at the lateral side 50 of the person's upper garment.

Next, in FIG. 4A, the same strap 110 is then threaded through an eyelet 210 that is part of, or attached to the flat portion 200.

In FIG. 4B, the other end of the same strap 110 continues from the eyelet 210 to be further attached to another portion 52 at the side of the person's upper garment.

Thus, in the example of FIGS. 4A and 4B, the strap 110 is arranged in a V-shaped configuration. Each of the ends of the arms of the V-shape are attached to points 51, 52 on the lateral side of the person's upper garment. The angular vertex of the strap passes through the eyelet 210 which, in turn, is connected to the flat portion 200 that is underneath the mattress 10.

In FIG. 3AA, the person is shown lying in the front-sleep position with his spine or body axis 13A generally parallel to the edge of the bed. The V-shaped strap arrangement of the strap is such that the actual strap runs freely through the eyelet 210. Thus, when the person's body axis is generally parallel to the edge of the bed, both the arms of the V-shape are roughly equal in length. However, if the person were to shift position slightly such that his body axis 13B were to be slightly askew from the parallel arrangement, the V-shaped strap arrangement can adjust to this slightly skewed orientation 13B, in the sense that the strap 110 would move through the eyelet 210 such that one of the arms would become longer at the expense of the other arm becoming shorter, so that the two arms of the V-shape would not be equal to each other.

Other modifications may provide a single connection between one point on the upper garment, and one point of the flat portion 200.

### Adjustability

In effect, the strap 110 functions as a leash, having a leash-length, that defines the outermost extent to which the person is permitted by the leash to move or roll away from the edge of the bed and the flat portion 200 beneath the mattress. For instance, a shorter leash length permits less rolling, compared to a longer leash length that permits a greater degree of rolling. Whatever the leash length of the arrangement selected by the user, however, the leash length cannot be of such a length as to allow the person to roll from the initial front-sleep position into the prohibited back-sleep position.

With regard to the length of the straps during use, it is appreciated that, in the preferred embodiment, one size cannot suit every circumstance. Therefore, the strap 110 includes leash-length-adjustment-means that allows length-adjustment of the leash-length of the person-to-anchor-connector-means. The one or more straps are thus adjustable in length.

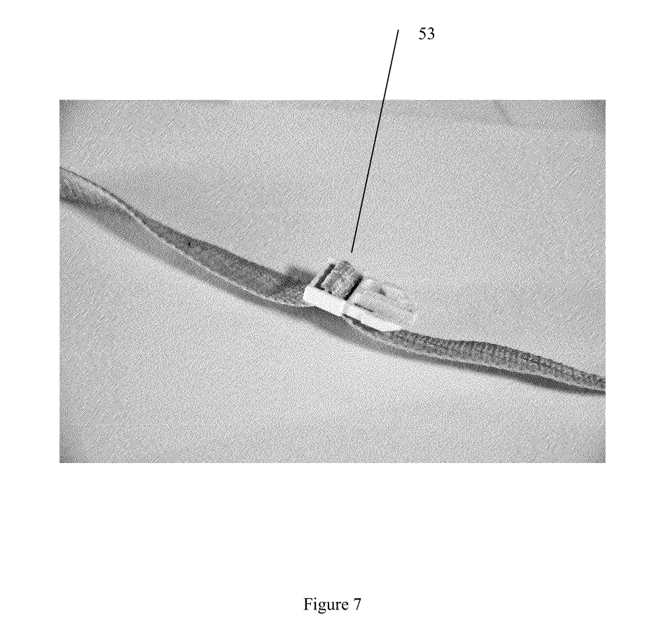

In the embodiment of FIGS. 4A and 4B, the leash-length-adjustment-means is in the form of a length-adjustor buckle 53, shown in FIG. 7. The adjustor buckle 53 allows the person to alter the overall length of the strap 110. The shorter the length, the less rolling that is permitted; the longer the length, the greater amount of rolling permitted.

The strap 110 is thus able to be arranged to have sufficient length to restrict the person to sleeping in the initial front-sleep position, and to be arranged so as to have insufficient length that would otherwise permit the person, from the initial front-sleep position, to subsequently adopt a back-sleep position.

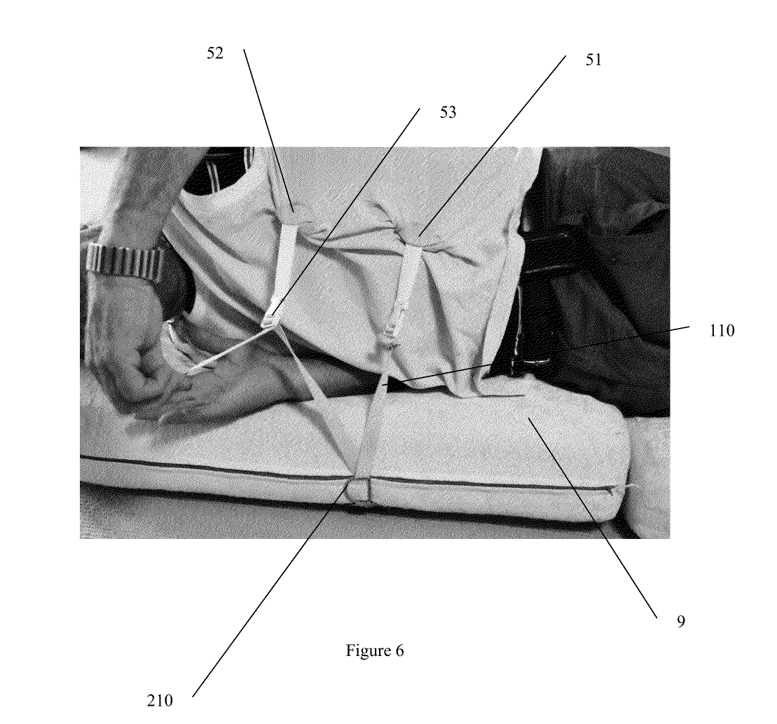

In use, the person lies down on the bed or mattress close to an edge of the bed. The person uses the strap 110 to connect himself to the flat portion 200 that is underneath the mattress. Then, in FIG. 6, the person uses the adjustor buckle 53 to adjust the length of the strap 110 so that it prevents him from fully rolling away from the edge into the back-sleep orientation. When the length is properly adjusted in this manner, the appropriate length allows a degree of freedom for the person to move around, but importantly, is of insufficient length to allow the person to roll away from the edge into the back-sleep position.

The notion of length adjustability of the straps should be construed broadly to cover an adjustment of the span of the strap between its connection point with the person, and its anchoring point. For example, in the embodiment of FIG. 4A, the physical dimension of the single strap 110 does not change, but rather, the adjustment of the adjustor buckle causes the overall leash-length, that is created by the strap, to be made shorter or longer. In other words, the notion of length does not imply that there can be only a single strip. For example, in other embodiments, a web of material can be used which can be adjusted to offer variations in length between the upper garment and the anchor.

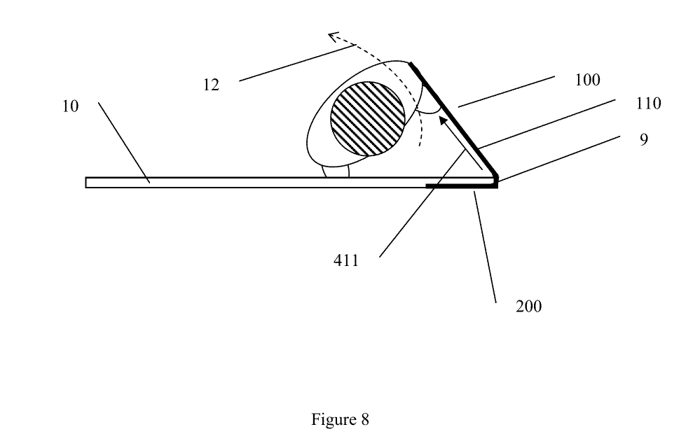

In other embodiments, the bed-means may be in the form that is thinner than a conventional mattress, for instance, a sleeping mat or close-cell foam mat used for camping, such as in FIG. 8.

In the embodiment of FIG. 5, the upper garment 54 is designed so that the it can be worn with either side of the garment facing the person's chest or front. This allows the person to select whether to attach the strap device 100 to either his right or left lateral side, depending on which side of the bed he sleeps.

The embodiments have been advanced by way of example only, and modifications are possible within the scope of the invention as defined by the appended claims.

In this specification, where the words comprise or comprises or derivatives thereof are used in relation to elements, integers, steps or features, this is to indicate that those elements, steps or features are present but it is not to be taken to preclude the possibility of other elements, integers, steps or features being present.

1. A device for preventing or lessening the likelihood of a person, prone to snoring and/or sleep apnoea, from experiencing the same, comprising:
a back-sleep-preventer adapted to ensure that, in use when a person lies on a bed-means initially in a front-sleep position and proximate an edge of the bed-means, the back-sleep-preventer is adapted to be arranged in a manner to substantially restrict the person to the front-sleep position to the extent of preventing the person from thereafter rolling away from the edge of the bed-means to adopt a back-sleep position such that, if the person is prone to snoring and/or sleep apnoea, then being prevented from adopting the back-sleep position by the back-sleep-preventer is able to prevent or at least lessen the likelihood of person experiencing snoring and/or sleep apnoea, wherein the back-sleep-preventer includes:
anchor-means able to releasably secure at least part of the back-sleep-preventer to the bed-means; and
person-to-anchor-connector-means adapted to connect the person or the person's upper garment to the anchor-means in order to substantially restrict the person to the front-sleep position in said aforesaid manner;
and wherein, in use when the person gets off and moves away from the bed-means, with the person-to-anchor-connector-means still connecting the person or the person's garment to the anchor-means, the anchor-means is able to release the at least part of the back-sleep-preventer from the bed-means.

2. A device of claim 1 wherein, in use when the bed-means includes a mattress or mat, the anchor-means is adapted to be inserted underneath the mattress or mat in order to substantially resist the pull of the person's rolling motion on the mattress or mat to restrict the person to the front-sleep position.

3. A device of claim 2 wherein the anchor-means includes weight-enhanceable-anchoring-means such that, in use when the person is lying on the mattress or mat with the person's body weight acting on the weight-enhanceable-anchoring-means, the weight-enhanceable-anchoring-means is enhanced in its ability to act as an anchor to the extent that the person's body weight and the weight of the bed-means makes it more difficult for the anchor-means to be disconnected from the bed-means.

4. A device of claim 3 wherein the weight-enhanceable-anchoring-means includes a flat portion that is adapted, in use, to be inserted underneath mattress or mat such that, in use when the person lies on the mattress or mat in a position that is above the flat portion, the person's body weight and/or the mattress weight are able to press on the flat portion thereunderneath to enhance the ability of the flat member to resist being pulled out from beneath the mattress or mat.

5. A device of claim 4 wherein, in use when the person gets off and moves away from the bed-means, with the person-to-anchor-connector-means still connecting the person or the person's garment to the flat portion, the flat portion is able to be pulled out from underneath the mattress or mat in order to release the at least part of the back-sleep-preventer from the bed-means.

6. A device of claim 5 wherein the person-to-anchor-connector-means connects to a lateral side of the person or of the person's garment.

7. A device of claim 5 wherein the person-to-anchor-connector-means connects to a lateral side of the person's upper garment.

8. A device of claim 5 wherein the person-to-anchor-connector-means is able to be arranged to have sufficient length to restrict the person to sleeping in the initial front-sleep position, and to be arranged so as to have insufficient length that would otherwise permit the person, from the initial front-sleep position, to subsequently adopt a back-sleep position.

9. A device of claim 5 wherein the person-to-anchor-connector-means functions as a leash having a leash-length that defines the outermost extent to which the person is permitted by the leash to move or roll away from the edge of the bed-means and the anchor-means.

10. A device of claim 9 wherein the person-to-anchor-connector-means includes leash-length-adjustment-means that allows length-adjustment of the leash-length of the person-to-anchor-connector-means.

11. A device of claim 5 wherein the person-to-anchor-means-connector-means includes one or more straps that are adjustable in length.

12. A method of preventing or lessening the likelihood of a person prone to snoring and/or sleep apnoea from experiencing the same, the method including:
restricting a person to a front-sleep position, when the person lies on a bed-means initially in a front-sleep position and proximate an edge of the bed-means, by arranging a back-sleep-preventer in a manner to substantially restrict the person to the front-sleep position to the extent of preventing the person from thereafter rolling away from the edge of the bed-means to adopt a back-sleep position such that the person being prevented from adopting the back-sleep position by the back-sleep-preventer is able to prevent or at least lessen the likelihood of person experiencing snoring and/or sleep apnoea,
wherein the method includes using person-to-anchor-connector-means of the back-sleep-preventer to connect the person or the person's upper garment to anchor-means of the back-sleep-preventer;
and also includes using the anchor-means to releasably secure at least part of the back-sleep-preventer to the bed-means in order to substantially restrict the person to the front-sleep position in said aforesaid manner, such that, when the person gets off and moves away from the bed-means, with the person-to-anchor-connector-means still connecting the person or the person's garment to the anchor-means, the method includes the anchor-means releasing the at least part of the back-sleep-preventer from the bed-means.

13. A method of claim 12 wherein the bed-means includes a mattress or mat, and the method includes inserting the anchor-means underneath the mattress or mat to releasably secure at least part of the back-sleep-preventer to the bed-means.

14. A method of claim 13 wherein the anchor-means includes weight-enhanceable-anchoring-means and wherein the method includes the person lying on the bed-means with the person's body weight acting on the weight-enhanceable-anchoring-means to allow the weight-enhanceable-anchoring-means to enhance its ability to act as an anchor to the extent that the person's body weight and the weight of the mattress makes it more difficult for the anchor-means to be disconnected from the bed-means.

15. A method of claim 14 wherein the weight-enhanceable-anchoring-means includes a flat portion, and wherein the method includes inserting the flat portion underneath the mattress of the bed-means such that the person lying on the mattress in a position above the flat portion causes the person's body weight and/or the mattress weight to press on the flat portion thereunderneath to enhance the ability of the flat member to resist being pulled out from beneath the mattress.

16. A method of claim 15 wherein, when the person gets off and moves away from the bed-means, with the person-to-anchor-connector-means still connecting the person or the person's garment to the flat portion, the method includes pulling the flat portion out from underneath the mattress or mat in order to release the at least part of the back-sleep-preventer from the bed-means.

17. A method of claim 12 wherein the method includes connecting the person-to-anchor-connector-means to a lateral side of the person, or to a lateral side of the person's upper garment.

18. A method of claim 12 wherein the method includes arranging the person-to-anchor-connector-means to have sufficient length to restrict the person to sleeping in the initial front-sleep position, and arranging the person-to-anchor-connector-means to so as to have insufficient length that would otherwise permit the person, from the initial front-sleep position, to subsequently adopt a back-sleep position.

19. A method of claim 12 wherein the method includes using the person-to-anchor-connector-means as a leash having a leash-length that defines the outermost extent to which the person is permitted by the leash to move or roll away from the anchor-means.
